(19) United States
(12) Patent Application Publication
(10) Pub. No.: US 2002/0147137 A1
(43) Pub. Date: Oct. 10, 2002

(54) Medical use of alpha1-acid glycoprotein (AAG) or orosomucoid

(75) Inventors: Konstantin Hiotakis, Graz (AT); Herwig Reichl, Ilz (AT)

Correspondence Address: Mark B. Wilson, FULBRIGHT & JAWORSKI L.L.P., 600 Congress Avenue, Suite 2400, Austin, TX, 78701, US

(73) Assignee: Hamosan Erzeugung Pharmazeutischer Grundstoffe Gesellschaft M.B.H., Graz (AU)

(21) Appl. No.: 09/978,317

(22) Filed: Oct. 15, 2001

Related U.S. Application Data
(63) Continuation of application No. PCT/AT00/00090, filed on Apr. 14, 2000.

(30) Foreign Application Priority Data
Apr. 14, 1999 (AU) A 667/99

(51) Int. Cl. A61K 38/17; A61K 38/38

(52) U.S. Cl. 514/8; 514/21

(57) ABSTRACT

Disclosed is the use of α1-acid glycoprotein (AAG) for the preparation of a medicament for influencing and accelerating drug withdrawal, as well as a kit comprising α1-acid glycoprotein and a plasmapheresis set for further plasma exchange with plasma and/or serum albumin solution.

The invention relates to a new medical use of α1-acid glycoprotein (AAG) or orosomucoid.

α1-acid glycoprotein (AAG) or orosomucoid is a protein occurring in plasma and having a molecular weight of approximately 40,000 and an isoelectric point of 2.7. It is the most soluble and most stable of all plasma proteins, which probably is due to its extremely high content of carbohydrates of approximately 40% (30-50%).

AAG consists of one single polypeptide chain having 183 amino acids and comprises 2 disulfide bonds. In the first half of the peptide chain, five carbohydrate chains are located, which consist of about 14% hexoses, 14% hexosamines, 1% fucose and 11-15% N-acetyl neuraminic acid (sialic acid). AAG occurs in different forms (2 A, 1 S, 1 F1) both as regards the polypeptide chair and as regards the carbohydrate chains.

The properties and biological functions of α1-acid glycoprotein (AAG) or orosomucoid have been reviewed by Schmid (in “The Plasma Proteins: Structure Function and Genetic Control”, Vol.1 (1975), Academic Press, Ed. Frank A. Putnam, 2^{nd} Edition, pp. 193-228) and Kremer et al. (Pharmacological reviews 40 (1988), pp. 1-47).

In medicine, a positive effect of α1-acid glyoprotein (AAG) in inflammatory reactions has been described (Denko et al., Agents and Actions 15, 5/6 (1984), 539-540). Libert et al. (J. Exp. Med. 180 (1994), 1571-1575) showed that a similar indication is the prevention of septic shock in connection with the effect of TNF-α or lipopolysaccharides Muchitsch et al. (WO 98/40087) showed “the use of human α1-acid gylcoprotein for preparing a pharmaceutical preparation” for treating disturbances of circulation and of microcirculation, respectively, of the non-inflammatory type.

Also described is the role of α1-acid glycoprotein in the transport of mainly basic medicaments in plasma (cf. Kremer et al.). α1-acid glycoprotein, in addition to human serum albumin (HSA), thus functions as one of the most important transport proteins, the charging state of the medicament being decisive for its binding: the transport of basic substances is preferred by 60 1-acid glycoprotein, thus e.g. methadone, diisopyramide, dipyridamol, lignocaine, progesterone, warfarin, chlorpromazine, quinidine, etc. Accordingly, AAG has also been suggested for an acute detoxification in case of drug poisoning, e.g. for the treatment of overdoses of quinine, lignocaine, propanulate or tricyclical anti-depressive agents such as amitriptyline, desipramine or nortriptyline (WO97/32893). such detoxification treatments utilise the strong binding of such medicaments to AAG, yet they have the disadvantage that they markedly prolong the actual elimination of the medicaments from the body. This o.a. has also been described for AAG.

Hervé et al. (Pharmacogenetics 6:5 (1996), 403-415) maintain that α1-acid glycoprotein must have at least 2 separate binding sites for ligands, whereas in earlier publications only one binding site had been assumed.

It has generally been known that in the treatment of drug diseases, in addition to the aspect of an acute medical detoxification of a patient, also the withdrawal treatment of the patient constitutes a substantial problem. In the withdrawal therapy relating to opiates, or also of substitute drugs, such as methadone (for instance in a drug substitution therapy methadone (L- or DL-methadone) is administered instead of heroin), serious physical withdrawal symptoms occur in addition to the psychical and psychosocial problems of such a change. These physical withdrawal symptoms by themselves make it advisable to maintain the patient under intensive medical care until such symptoms have subsided, which is hardly achieved within less than one week.

Since methadone binds to both human serum albumin (HSA), and to α1-acid glycoprotein (AAG) (cf. e.g. Romach et al. in Clin. Pharmacol. Ther. 29:2 (1981), 211-217), it is assumed that since methadone does not only occur freely within the plasma but also bound to these proteins, the plasma exchange thus will remove both fractions.

Garrido et al. (J. Pharm Pharmacol. 48:3 (1996) 281-284) showed an increase in the AAG concentration in plasma after an oral administration of morphine to rats, as well as during acute inflammations (Gomez et al., Gen. Pharmacol 26:6 (1995) 1273-1276). In both instances also the methadone binding changed, and therefore the authors suggest an adaptation of the methadone dose both in a withdrawal procedure and if acute inflammations occur.

Since during the withdrawal treatment in addition to the psychosomatic and psychological parameters of a patient, primarily the return to normal of the endogenous functions of the body which had been changed by the drug abuse are of primary importance, means which serve for an acute detoxification cannot be used, since the withdrawal proper can be started in a stabilized drug abuse patient at the earliest, but by no means during an acute drug poisoning.

According to an investigation by Øie and Fiori (1985) on isolated perfused rat livers, the elimination of drugs which bind to HSA and α1-acid glycoprotein is differently influenced by the addition of these proteins to the perfusate:

Albumin has the ability to accelerate the elimination of drugs irrespective of whether the latter bind to albumin or not.

By α1-acid glycoprotein on the other hand, the elimination of drugs capable of being bound thereby is slowed down, whereas it has no influence on the elimination of nonbinding drugs.

In the withdrawal therapy of a methadone addict, one of the inventors has practised the method of plasma exchange (PE) for assisting and accelerating the removal of methadone from the body, i.e., blood was taken from the patient, the cellular components were separated and re-infused into the body, and instead of the patient's own plasma portion, the plasma of a donor was supplied. This accelerated the withdrawal, as can be taken from the following data.

| Substance: | | Day 1 | Day | Day 3 | Day | Day | Day | Day |
|---|---|---|---|---|---|---|---|---|
| before | 1. PE | (1.PE) | 2 | (2.PE) | 4 | 5 | 6 | 7 |
| methadone | 16 | 5 | 2.7 | 0.9 | 0.9 | 1.5 | 1.6 | 1.1 |
| methabo- | | | | | | | | |
| lite | | | | | | | | |
| methadone | 15 | 3 | 1.9 | 0.5 | 0.5 | 0.4 | 0.3 | 0.5 |
| thebaine | 7 | 0 | 0 | 0 | 0 | 0 | 0 | 0 |

(Substances measured in urine, μg/ml)

Description of the treatment, cf. Example 1.

This result showed that by the plasma exchange, methadone is withdrawn more rapidly from the body so that the duration of the withdrawal therapy could be shortened and the painful effects could also be reduced.

The method of plasmapheresis, although industrially used for the recovery of plasma, nevertheless is quite complicated, expensive, and complex also from the medical point of view. Since in the present treatment regimen it was used twice, and the patient additionally had to get two plasma donations, whereas the patient's plasma had to be discarded (drug addiction is a reason for excluding blood and plasma donors), this part of the treatment must be judged as being very cost intensive. Therefore, alternatives have. been sought.

It has been suggested to supply a 5% stabilized physiological albumin solution to the patient instead of plasma, which would also have advantages from the virological point of view. However, to exchange significant portions of the plasma again a multiple plasmapheresis with removal of the patient's own plasma and replacement thereof, this time with HSA solution, would have been required.

Thus, it is an object of the present invention to provide a medicament with which drug withdrawal can be decisively improved as compared to the measures known from the state of the art. Such a medicament therapy should at least involve a comparable, if not even better, positive influence on, and acceleration of, the withdrawal process than plasmapheresis alone, and at the same time maintain the patient in as positive a physical and mental condition as possible.

According to the invention, this object is achieved by using AAG for preparing a medicament for influencing and accelerating drug withdrawal.

According to the invention, surprisingly it has been found that with the use of AAG administration during drug withdrawal, particularly during a heroin or methadone withdrawal, not only a clear acceleration of the reconstitution of the “normal state” of the patient will occur but that also an intensive recovery phase is enabled without any withdrawal symptoms which has decisive advantages even over the expensive complex and risky plasmapheresis treatment.

This was surprising also because, according to the course of the first withdrawal with a plasma exchange, as well as according to the statements given in the literature, HSA was to be considered as an essential protein for such a withdrawal modulation.

Contrary to the opinion described in the prior art, in particular by Øie and Fiori (1985), according to the invention it could be shown that the concern that drugs such as methadone due to their binding to AAG could be eliminated only at a slower rate was unfounded.

In fact, the effect according to the invention was first found in a comparative assay with which possible negative influences of AAG in the previously described methods (plasma contains 0.3-1.1 mg AAG/ml) were to be excluded. In fact, however, the patients who were subjected to this negative control (whom a sterile isotonic AAG solution had been infused prior to carrying out the plasmapheresis) showed the most favorable results during drug withdrawal.

Quite surprisingly, after a short intensive crisis which probably was due to too rapid an infusion of the 2^{nd} partial amount (α1-acid glycoprotein is an acute phase protein!), an intensive recovery phase without any withdrawal symptoms followed. The measurement of the methadone metabolites in urine showed, starting from 760 μg absolute at admission, a rise to 4700 μg after 11 hours (=7 hours after the 1^{st} administration and 1 hour after the 2^{nd} administration of α1-acid glycoprotein), and an equally rapid decrease to 1200 μg, 700 μg, and 100 μg after 13, 17 and 24 hours. During the following days, the value oscillated between 250 and 0 μg.

After this surprising change, no further plasma exchange and HSA administration were carried out. The patient felt well and did not show any physical withdrawal symptoms, the only problem was nicotine-withdrawal.

After a total of three days, treatment could be terminated. When comparing the course of the two treatments, it is striking that with the 2^{nd} patient, the withdrawal symptoms—which when compared to common withdrawals were much lighter and shortened—of the 1^{st} treatment could be almost completely avoided. Also the single plasmapheresis and hemodilution could have been done without, since they were only a matter of the original treatment plan.

The preparation of α1-acid glycoprotein has been known for decades (Hao et al., Biocimica et Biophysica Acta 322 (1973), 99-108; WO 95/07703; WO 97/32893); at the end of a Cohn's plasma fractionation process, it is easily available in large amounts. It may also be recovered by genetical engineering methods.

The splitting of α1-acid glycoprotein into subclasses is technically feasible and has been described in the literature.

Preparations of individual subclasses of α1-acid glycoprotein also fall within the scope of this invention, just as α1-acid glycoprotein recovered by genetical engineering methods.

According to the invention, the preparation is produced as an isotonic storage-stable infusion solution and is delivered as such or as a lyophilisate.

The dose for the respective indication will depend on the patient's general state. The total amount of 10 mg to 1 g/kg, preferably 50-250 mg/kg body weight, may either be administered as a single dose or distributed to several doses.

The application may be effected in any manner, yet diluted infusions i.v. are preferred.

Preferably, according to the invention human AAG prepared from plasma is used, yet also recombinant human AAG has advantageous properties as compared to other AAG preparations which can be used according to the invention, in particular if it has been produced in cells which are capable of delivering a suitable glycosilation pattern on AAG (e.g. mammalian cells).

Preferably, pharmaceutical preparations containing more than 10% of pure α1-acid glycoprotein, in particular more than 75%, at best such, in which more than 90% of the total protein are present as α1-acid gylcoprotein, are used. The rest may consist of human serum albumin (HSA) and other plasma proteins.

Preferably, the pharmaceutical preparation is stabilized, i.e., for increasing its storage stability and its stability during a heat treatment, stabilizers in particular sodium caprylate, are admixed.

It is also recommendable to treat the pharmaceutical preparation for an inactivation and/or depletion of viruses, in particular by heating and/or by virus filtration. Other treatment methods may be the solvent/detergent method (S/D), perchloric acid treatment, but also treatments with organic solvents.

To inactivate prions, a new group of extremely resistant pathogens, other methods, in particular the Haemosafe method described in EP 0 530 173, may be used.

The invention will now be explained by the following examples and diagrams, without, however, being restricted thereto. In particular, in case of a poisoning by basic drugs, a treatment with α1-acid glycoprotein seems to be very promising.

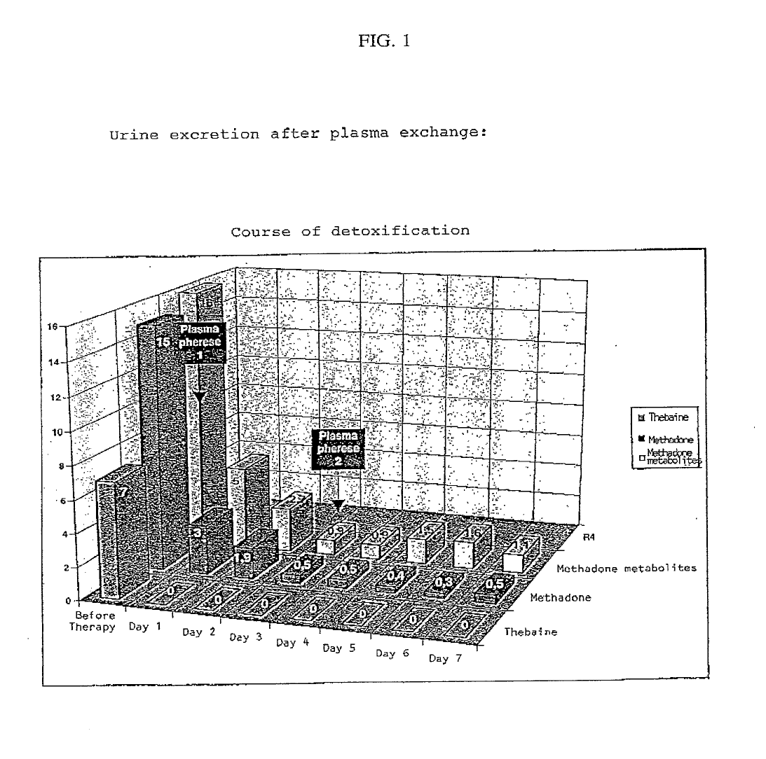

FIG. 1 shows a drug withdrawal by means of plasma exchange and

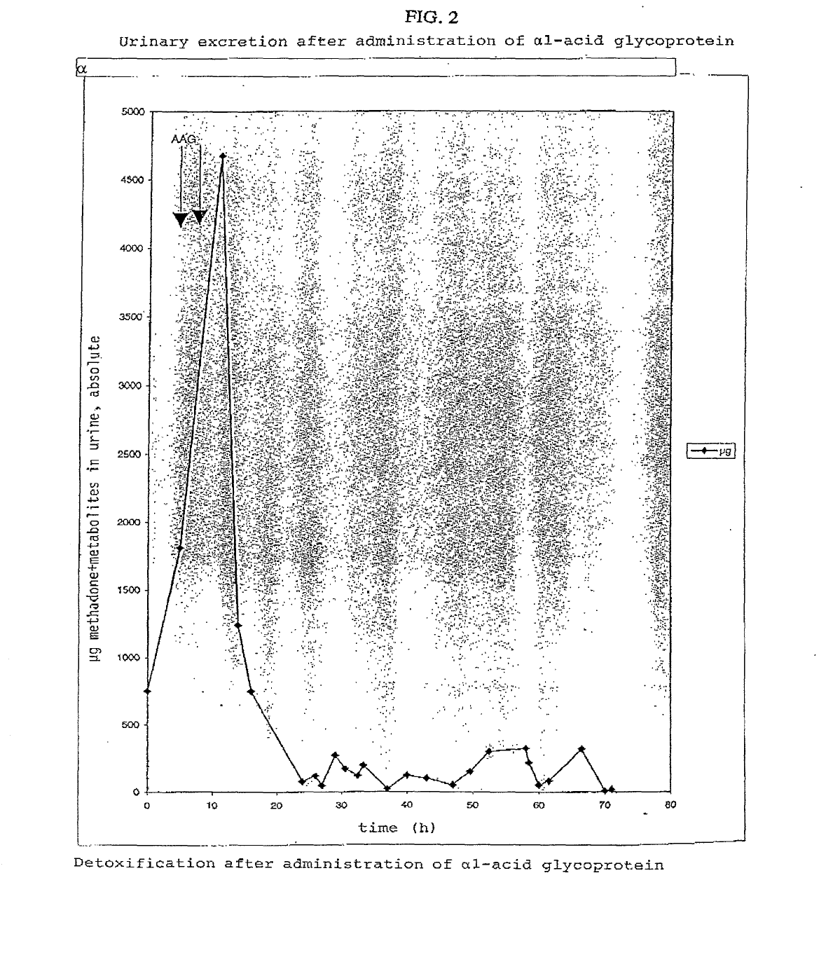

FIG. 2 shows a drug withdrawal by means of AAG.

### EXAMPLE 1

### Withdrawal by Means of Plasma Exchange (Comparative Assay)

Preliminary Arrangements:

The patient appears in the intensive care unit early in the morning after having taken the methadone dose (the drug is taken because drug addicts are highly sensitive to pain and very restless after withdrawal of their drug).

Consent

The patient agrees in writing to a withdrawal therapy and only then will be admitted to the intensive care unit. In the intensive care unit, a central venous catheter will be applied and blood will be taken.

Method

To minimize the methadone and methadone metabolites the plasmapheresis was necessary. The patient was 2 x connected to the plasmapheresis (1^{st} and 3^{rd} day). Every day the qualitative and quantitative determination of methadone and methadone metabolites was measured (cf. FIG. 1) and precisely balanced. Besides, the coagulation parameters were measured because of the hypercoagulability.

Course:

When the patient has been admitted to the intensive care unit and the cava catheter has been applied and a complete monitoring has been started, the plasmapheresis begins 3 hours thereafter, the patient begins to sweat, has sensations of cold and hot, the blood pressure rises and the patient has a subjective urge to urinate; the symptoms, however, will cease without any therapy, loving conversation with the nursing staff will suffice. Within one hour the symptoms will cease without any therapy involving medicaments. After further 3 hours, the patient's state will be stable, yet it will not be possible to urinate spontaneously. For this reason, a urinary catheter will be set. After further 3 hours, the subjective and objective symptoms will slightly increase, and therefore the therapy midazolame 0.4 μg/kg/h and 0.1 mg/kg/d of clonidine will be started by means of a perfusor. One hour later the patient will not exhibit any symptoms anymore, the urinary catheter will be removed, and spontaneous urination is possible. Since the patient eats only little, he will be fed parenterally in addition.

2^{nd} Day:

The patient feels well, is co-operative, and due to the above indicated therapy, because of the risk of thrombosis (Hb 16.1 g/dl and HTK 45.9%), a blood-letting of 300 ml whole blood is carried out.

3^{rd} Day:

After the onset of the plasmapheresis, withdrawal symptoms occur, the patient becomes fidgety with a tendency to cramps, the urge to urinate, a slight respiratory insufficiency (apnoe phases), hyperventilation and, finally, the onset of confusion.

The sensitivity to pain is so extremely increased that any grip on the patient, any touch between patient and nursing staff or a doctor is experienced as painful by the patient. This condition is improved with an appropriate therapy. The therapy administered by a perfusor and consisting of Midazolam and Clonidin is increased to twice the dosage, and the patient has to get a bolus of 5 mg of Midazolam in this critical phase.

This slight hypoxia was corrected with an oxygen supply using a mask. Despite all the above mentioned measures of intensive medical care, the patient is restless, sweaty, disoriented and additionally contabulated. This dangerous condition lasted for approximately 10 hours, with the constant attention of the nursing staff and doctors. After these difficult hours, the patient's state became stable, and the patient could sleep through the night without any striking symptoms.

4^{th} Day:

The patient feels well, is co-operative, is oriented and has had an enjoyable and joyful breakfast. The Clonidin-Hidazolam supply can be reduced again to the original dosage. Until noon, the patient's state becomes so good that the patient asks to go for a walk in the park of the hospital, of course accompanied by a member of the nursing staff.

5^{th} Day:

The patient's general state is a very good one, the patient is mobile, completely normal and takes walks in the park. The parenteral therapy, Midazolam and Clonidin, has been changed to an oral therapy. Every 6 hours, Clonidin 0.5 mg and Midazolam 7.5 mg. The additional parenteral feeding has been stopped, and the cava catheter is removed.

6^{th} Day:

The state is unchanged, the oral medicamentous therapy is once more reduced; every 4 hours, Clonidin 0.075 mg and Midazolam 4.5 mg.

7^{th}, 8^{th}, 9^{th} Day:

The patient's general state is unchanged and a very good one. The patient says that after 12 years for the first time he feels like born anew.

On the 10^{th} day the patient is dismissed from the intensive care unit and of his own free will goes to Vienna for further treatment.

Course of detoxification

| | Before | Day | Day | Day | Day | Day | Day | Day |
|---|---|---|---|---|---|---|---|---|
| | Therapy | 1 | 2 | 3 | 4 | 5 | 6 | 7 |
| thebaine | 7 | 0 | 0 | 0 | 0 | 0 | 0 | 0 |
| methadone | 15 | 3 | 1.9 | 0.5 | 0.5 | 0.4 | 0.3 | 0.5 |
| methadone | 16 | 5 | 2.7 | 0.9 | 0.9 | 1.5 | 1.6 | 1.1 |
| metabolites | | | | | | | | |

| After Discharge | Day 17 | Day 24 | Day 31 |
|---|---|---|---|
| thebaine | 0 | 0 | 0 |
| methadone | 0 | 0 | 0 |
| methadone metabolites | 0 | 0 | 0 |

Data in μg/ml urine.

Corresponding diagram: FIG. 1

### EXAMPLE 2

### Methadone Withdrawal by Administration of α1-Acid Glycoprotein (According to the Invention)

9/19/97/ZO

Last methadone intake at 6:30 a.m.: 50 mg.

The patient's circulation is stable, the patient is co-operative, a venous cannula and a peripheral venous catheter on the left side are set. Immediately after admission, beginning of sedation with Dormicum 0.4 mg/kg/d, Clonidin 0.02 mg/kg/d. Dormicum bolus administration at admission.

At 10 a.m., administration of 3 grams of α1-acid glycoprotein in 500 ml of infusion solution. Starting from 12 o'clock, first symptoms with slight sweating, otherwise no problems so far. At 1:50 p.m., blood-letting (300 ml) and hemodilution: 500 ml of own blood taken. After separation into ery and plasma, the erythrocytes are returned into the patient. Volume substitution with Elohaest, human albumin and electrolyte solution.

From 4:05 p.m. to 4:15 p.m., renewed hemodilution: 400 ml of own blood was drawn. After separation, the erythrocytes are re-transfused again. volume substitution again with glucose, electrolyte and 5% human albumin solution.

After the administration of 6 grams of α1-acid glycoprotein in merely 150 ml of infusion solution at 4:30 p.m., symptoms of an acute reaction, similar to an allergy (prickly sensation in hands and feet, starting swelling, generalizing exanthema, bronchospasm, swelling of lids and lips) set in. Rapid improvement within half an hour with an appropirate therapy. Afterwards the patient is free from symptoms and free from any discomforts, with a stable circulation. In the evening and night hours, the patient is tired, the prescribed sedation has to be reduced temporarily. Intake of food is mostly per os and partly parenterally. Urinating is without any problems and spontaneous.

9/20/97/Zo

The patient's circulation is stable, the-patient is free from fever, co-operative, no physical withdrawal symptoms. The patient's only problems are nicotine withdrawal. The Dormicum-Clonidin dosage is reduced to half the amount and only has to be slightly increased again during the night hours past midnight because of discomfort in the chest; subsequently, the patient is again free from any discomfort.

9/21/97/Zo

Except for an obstructed breathing through the nose, no particular physical withdrawal symptoms. Therapy with Clonidin and Dormicum is changed to oral (4×0.075 mg Catapresan and 4×7.5 mg Dormicum per os) with sufficient magnesium substitution in addition. Subjectively, the patient feels well and is free from any discomfort.

9/22/97/ZO

Removal of the peripheral venous catheter, continuation of the oral therapy, the patient is moved to the LNKH while being in a good general state and subjectively feeling well.

Diagram of urine excretion of methadone and its metabolites cf. FIG. 2. The values are immunologically measured by means of ADX analyser from Abbott and constitute the total values of methadone plus methadone metabolites per urine sample.

1. The use of human α1-acid glycoprotein for the preparation of a medicament for influencing and accelerating drug withdrawal.

2. The use according to claim 1, characterized in that a stabilized storable infusion solution is prepared.

3. The use according to claim 1 or 2, characterized in that a lyophilisate is prepared.

4. The use according to any one of claims 1 to 3, characterized in that a medicament comprising at least 10%, preferably more than 50%, in particular more than 90%, of α1-acid glycoprotein, based on the total protein, is prepared.

5. The use according to any one of claims 1 to 4, characterized in that the medicament furthermore comprises human serum albumin.

6. The use according to any one of claims 1 to 5, characterized in that the medicament is treated for the inactivation and/or depletion of viruses, in particular by at least one physical treatment, such as heat treatment and/or virus filtration.

7. The use according to any one of claims 1 to 6, characterized in that the medicament is treated for the inactivation and/or depletion of prions, in particular by at least one validated inactivation method.

8. The use according to any one of claims 1 to 7, characterized in that the medicament comprises at least one stabilizer, in particular sodium caprylate.

9. The use according to any one of claims 1 to 8, characterized in that the medicament is employed in combination with a plasma exchange.

10. A kit comprising α1-acid glycoprotein and a plasmapheresis set for further plasma exchange with plasma and/or serum albumin solution.
